(19) United States
(12) Patent Application Publication
(10) Pub. No.: US 2020/0000320 A1
(43) Pub. Date: Jan. 2, 2020

(54) ENDOSCOPE

(71) Applicant: HOYA CORPORATION, Tokyo (JP)

(72) Inventors: Yuki TAJIMA, Tokyo (JP); Kensei SOMEKAWA, Tokyo (JP)

(73) Assignee: HOYA CORPORATION, Tokyo (JP)

(21) Appl. No.: 16/484,270

(22) PCT Filed: Jan. 30, 2018
(86) PCT No.: PCT/JP2018/002917

§ 371 Date: Aug. 7, 2019

(30) Foreign Application Priority Data
Mar. 31, 2017 (JP) 2017-072398

(51) Int. Cl. A61B 1/00; A61B 1/07

(52) U.S. Cl. CPC A61B 1/00096; A61B 1/07; A61B 1/00163

(57) ABSTRACT

An endoscope is provided that obtains a favorable field of view. The endoscope includes: an illumination optical system that has an illumination window protruding from an end surface of an insertion unit; a cylindrical body that protrudes from the end surface; and an observation window that is arranged inside the cylindrical body and protrudes from the end surface, in which the illumination window and the observation window satisfy Formulas (1) and (2), and an intersection between a straight line, which connects an edge of a light incident surface of the observation window and an edge of an end surface of the cylindrical body, and an optical axis of the illumination optical system is located to be closer to the insertion unit than the end surface of the insertion unit.

C≥D (1)

E≥D (2).

### TECHNICAL FIELD

The present invention relates to an endoscope.

### BACKGROUND ART

A distal end portion of an endoscope is provided with an illumination window to emit illumination light and an observation window to observe a part illuminated with the illumination light (Patent Literature 1).

### CITATION LIST

### Patent Literature

Patent Literature 1: JP 2004-49793 A

### SUMMARY OF INVENTION

### Technical Problem

When the illumination light emitted from the illumination window is directly incident on the observation window, so-called flare occurs in an observation field of view, which hinders observation using the endoscope. An object of the present invention is to provide an endoscope to obtain a favorable observation field of view by preventing occurrence of flare.

### Solution to Problem

An endoscope includes: an illumination optical system that has an illumination window protruding from an end surface of an insertion unit; a cylindrical body that protrudes from the end surface; and an observation window that is arranged inside the cylindrical body and protrudes from the end surface. The illumination window and the observation window satisfy Formulas (1) and (2). An intersection between a straight line, which connects an edge of a light incident surface of the observation window and an edge of an end surface of the cylindrical body, and an optical axis of the illumination optical system is located to be closer to the insertion unit than the end surface of the insertion unit.

C≥D (1)

E≥D (2)

C is a maximum protruding amount of the observation window from the end surface of the insertion unit.

D is a maximum protruding amount of the illumination window from the end surface of the insertion unit.

E is the protruding amount of the edge of the light incident surface of the observation window from the end surface of the insertion unit.

### Advantageous Effects of Invention

It is possible to provide the endoscope to obtain the favorable field of view.

### BRIEF DESCRIPTION OF DRAWINGS

FIG. 1 is an exterior view of an endoscope.

FIG. 2 is an external view of an end surface of a distal end portion.

FIG. 3 is an explanatory view for describing a configuration of the distal end portion.

FIG. 4 is a cross-sectional view of the distal end portion taken along line IV-IV in FIG. 2.

FIG. 5 is an enlarged view of a part A in FIG. 4.

FIG. 6 is an explanatory view for describing a point L.

FIG. 7 is an enlarged view of a part B in FIG. 6.

FIG. 8 is a cross-sectional view of the distal end portion of FIG. 2 taken along line VIII-VIII.

FIG. 9 is a cross-sectional view of a distal end portion of a second embodiment.

### DESCRIPTION OF EMBODIMENTS

### First Embodiment

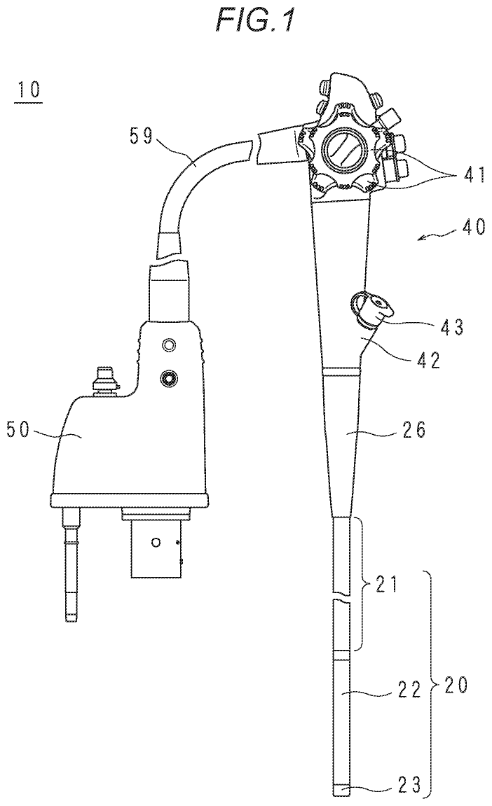

FIG. 1 is an exterior view of an endoscope 10. An endoscope 10 of the present embodiment is a flexible scope for a lower gastrointestinal tract. The endoscope 10 includes an insertion unit 20, an operation unit 40, a universal cord 59, and a connector unit 50. The operation unit 40 has a bending knob 41 and a channel inlet 42. A forceps plug 43 having an insertion port to insert a treatment tool or the like is fixed to the channel inlet 42.

The insertion unit 20 is long and has one end connected to the operation unit 40 via a bend preventing portion 26. The insertion unit 20 includes a soft portion 21, a bending portion 22, and a distal end portion 23 in the order from the operation unit 40 side. The soft portion 21 is soft. A surface of the soft portion 21 is a tube-shaped flexible tube. The bending portion 22 is bent according to an operation of the bending knob 41.

In the following description, a longitudinal direction of the insertion unit 20 is referred to as an insertion direction. Similarly, a side close to the operation unit 40 along the insertion direction is referred to as an operation unit side, and a side far from the operation unit 40 is referred to as a distal end side.

The universal cord 59 is long, and has a first end connected to the operation unit 40 and a second end connected to the connector unit 50. The universal cord 59 is soft. The connector unit 50 is connected to a video processor (not illustrated), a light source device, a display device, an air and water supply device, and the like.

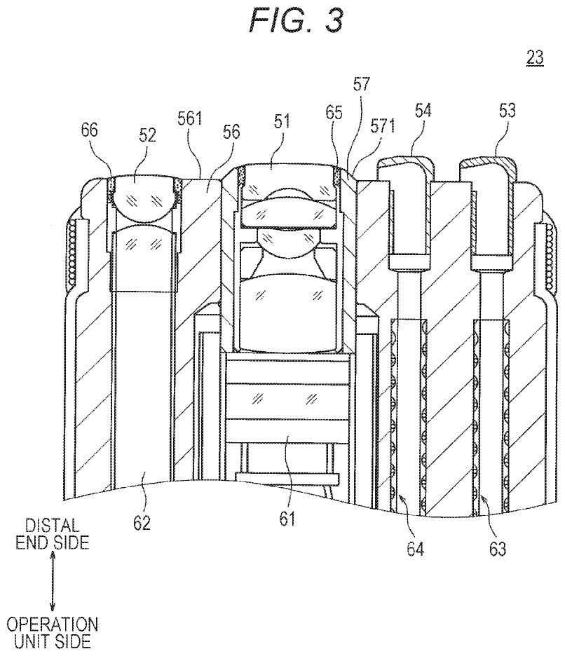

A fiber bundle 62 (refer FIG. 3), an air supply tube 63 (refer FIG. 3), a water supply tube 64 (refer FIG. 3), a cable bundle, and the like, which will be described later, are inserted inside the connector unit 50, the universal cord 59, the operation unit 40, and the insertion unit 20.

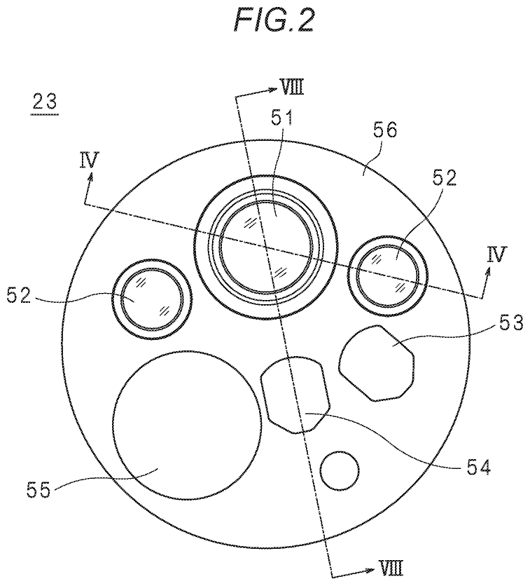

FIG. 2 is an external view of an end surface of the distal end portion 23. A distal end member 56 having a substantially cylindrical shape is coaxially fixed to the insertion unit 20 at the distal end portion 23. An observation window 51, two illumination windows 52, an air supply nozzle 53, a water supply nozzle 54, a channel outlet 55, and the like are fixed to a distal end of the distal end member 56.

Incidentally, FIG. 2 is an example of an appearance of the end surface of the distal end portion 23, and an arrangement of each member is not limited to FIG. 2. For example, the arrangements of the air supply nozzle 53 and the water supply nozzle 54 may be reversed. The illumination window 52 may be one or three or more.

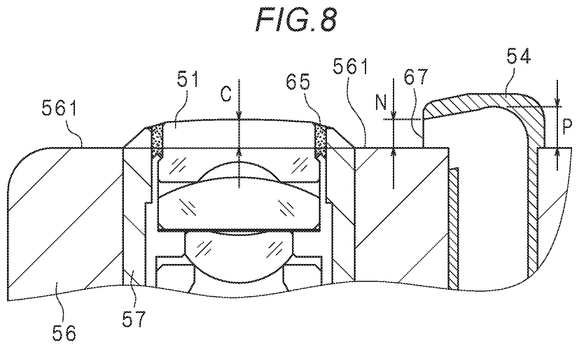

The observation window 51 is provided above a center of the distal end member 56 in FIG. 2. The illumination windows 52 are provided on the left and right of the observation window 51. The air supply nozzle 53 and the water supply nozzle 54 are provided with outlets 67 (see FIG. 8) facing the observation window 51 at the lower right of the observation window 51. The channel outlet 55 is provided at the lower left of the observation window 51.

FIG. 3 is an explanatory view for describing a configuration of the distal end portion 23. FIG. 3 is a composite cross-sectional view in which cross sections obtained by cutting the observation window 51, one of the illumination windows 52, the air supply nozzle 53, and the water supply nozzle 54 along the insertion direction are connected. In FIG. 3, the upper side is the distal end side, and the lower side is the operation unit side.

The illumination window 52 protrudes from a distal end surface 561 which is an end surface of the distal end member 56. The illumination window 52 is a convex lens as a distal end component of an illumination optical system that irradiates an observation target with illumination light emitted from a light source device. A radius of curvature of a front surface side of the illumination window 52, that is, an emission surface side of the illumination light is larger than a radius of curvature of a back surface side of the illumination window 52, that is, an incident surface side of the illumination light. The illumination window 52 is fixed to the distal end member 56 by an illumination bonding portion 66.

An end portion of the fiber bundle 62 is arranged on the operation unit side of the illumination window 52. The fiber bundle 62 is a bundle of multiple optical fibers. The end portion of the fiber bundle 62 is hardened by an adhesive and formed in a convex shape. The illumination light emitted from the light source device irradiates the distal end side of the insertion unit 20 through the fiber bundle 62 and the illumination window 52.

Shapes and arrangements of the fiber bundle 62 and the illumination window 52 illustrated in FIG. 3 are examples of the configuration of the illumination optical system. The configuration of the illumination optical system is appropriately selected so as to have a wide irradiation angle capable of illuminating the field of view of an observation optical system as will be described later.

The observation window 51 is an optical component on the distal end side of the observation optical system that observes the range illuminated by the illumination light. A front surface side of the observation window 51, that is, an incident surface side of observation light is a convex surface. A back surface side of the observation window 51, that is, a central portion on an emission surface side of the observation light is a concave surface. Therefore, the observation window 51 is a so-called meniscus lens.

Optical components such as a convex lens and a concave lens are arranged on the operation unit side of the observation window 51. An imaging element 61 is arranged at a composite focal position of the optical components. The observation optical system can realize an optical system to obtain a wide observation field of view using the meniscus lens for the observation window 51.

The observation window 51 is not limited to the meniscus lens. As the entire observation optical system, optical components having arbitrary shapes that can obtain desired optical performance according to the application can be used.

The imaging element 61 is connected to a video processor via a cable bundle. A video signal generated by the imaging element 61 is transmitted to the video processor via the cable bundle and displayed on a display device such as a liquid crystal display.

A cylindrical body 57 is arranged so as to protrude from the distal end surface 561. The optical components such as the observation window 51 are arranged inside the cylindrical body 57 in a state where the observation window 51 protrudes from the distal end surface 561.

A first chamfer 571 is provided at an outer periphery on the distal end side of the cylindrical body 57. The first chamfer 571 is a conical surface. An inner surface of the distal end side of the cylindrical body 57 and an outer edge of the observation window 51 are fixed to each other by an observation bonding portion 65. The observation bonding portion 65 and the cylindrical body 57 have a light shielding property, and prevent unnecessary light from entering the observation optical system from the lateral side.

Shapes and arrangements of optical components such as the observation window 51 illustrated in FIG. 3 are examples of the configuration of the observation optical system. The configuration of the observation optical system is appropriately selected according to the application and specification of the endoscope 10.

The water supply nozzle 54 has a substantially L-shaped cylindrical shape. The water supply nozzle 54 is connected to the water supply tube 64 via a through-hole provided in the distal end member 56. Water supplied from the air and water supply device is discharged from the water supply nozzle 54 toward the observation window 51 via the water supply tube 64.

The air supply nozzle 53 has a substantially L-shaped cylindrical shape. The air supply nozzle 53 is connected to the air supply tube 63 via a through-hole provided in the distal end member 56. Air supplied from the air and water supply device is discharged from the air supply nozzle 53 toward the observation window 51 via the air supply tube 63.

Heights of inner surfaces of the air supply nozzle 53 and the water supply nozzle 54 are lower on the side closer to the observation window 51 with respect to the distal end surface 561. As a result, it is possible to increase each momentum of water flow and air flow which are ejected from the air supply nozzle 53 and the water supply nozzle 54. The air supply nozzle 53 and the water supply nozzle 54 are used, for example, to clean the observation window 51 during an endoscopy.

The description will be continued returning to FIGS. 1 and 2. The channel inlet 42 and the channel outlet 55 are connected by a tube-shaped channel passing through each inside of the soft portion 21 and the bending portion 22. As a treatment tool (not illustrated) is inserted from the channel inlet 42, a distal end of the treatment tool can be caused to protrude from the channel outlet 55 to perform a procedure such as removal of a polyp.

A user of the endoscope 10 according to the present embodiment inserts the insertion unit 20 from the anus of a person to be examined.

The user guides the distal end of the insertion unit 20 to a target site while observing a captured image through the observation window 51. At a part where the large intestine is strongly bent, the user operates the bending knob 41 to bend the bending portion 22 and performs an operation such as twisting the insertion unit 20 so as to advance the distal end portion 23 toward the cecum. The soft portion 21 that has entered the inside of the large intestine is pushed against a wall of the large intestine and bent passively.

Incidentally, the endoscope 10 may be a flexible mirror for an upper gastrointestinal tract or a respiratory system. The endoscope 10 may be a rigid endoscope such as a laparoscope. There is a case where the endoscope 10 does not include the air supply nozzle 53, the water supply nozzle 54, the bending portion 22, and the like depending on the application and specification.

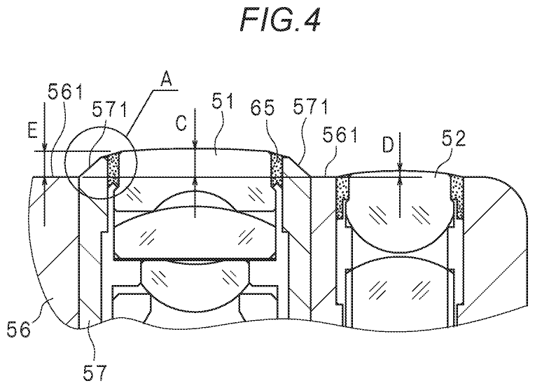

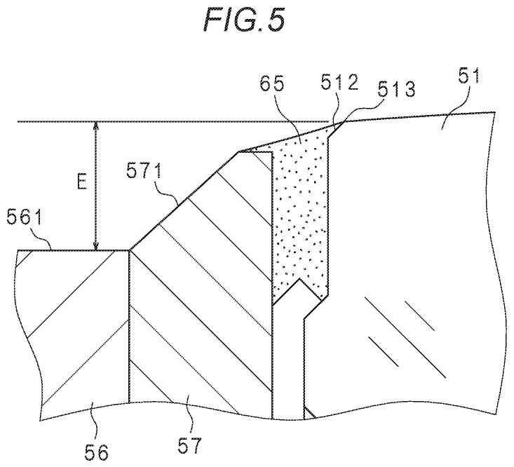

FIG. 4 is a cross-sectional view of the distal end portion 23 taken along line IV-IV in FIG. 2. FIG. 5 is an enlarged view of a part A in FIG. 4.

Symbols to be used in the following description will be described using FIGS. 4 and 5. A maximum protruding amount of the observation window 51 using the distal end surface 561 as a reference is indicated by a symbol C. A maximum protruding amount of the illumination window 52 using the distal end surface 561 as a reference is indicated by a symbol D. A protruding amount of an edge 513 of the light incident surface of the observation window 51 using the distal end surface 561 as a reference is indicated by a symbol E.

The edge 513 of the light incident surface of the observation window 51 will be described in more detail using FIG. 5. The edge 513 of the light incident surface means an edge on the distal end side of a second chamfer 512 provided at the edge of the observation window 51. The chamfer at the edge and side surfaces of the observation window 51 are covered with the observation bonding portion 65 and the cylindrical body 57 which have the light shielding property. Therefore, light does not enter the observation window 51 from these parts.

As illustrated in FIG. 4, the protruding amounts of the observation window 51 and the illumination window 52 satisfy Formulas (3) and (4).

C≥D (3)

E≥D (4)

C is the maximum protruding amount of the observation window 51 from the distal end surface 561 of the insertion unit 20.

D is the maximum protruding amount of the illumination window 52 from the distal end surface 561 of the insertion unit 20.

E is the protruding amount of the edge 513 of the light incident surface of the observation window 51 from the distal end surface 561 of the insertion unit 20.

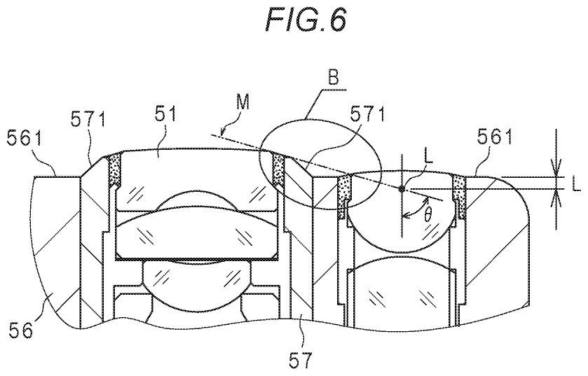

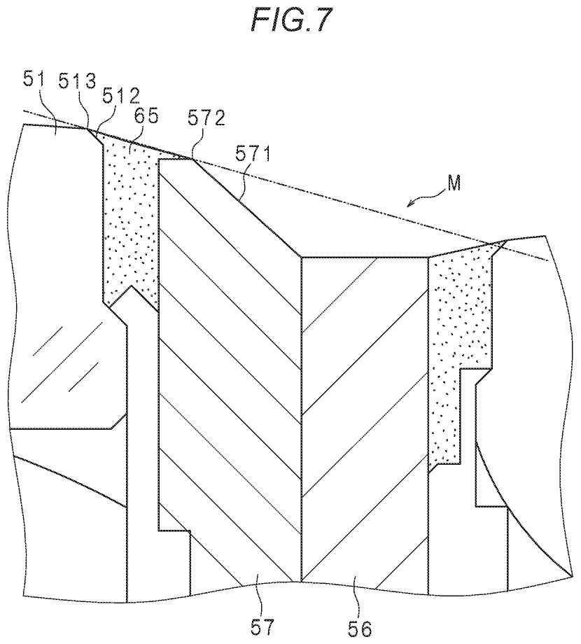

FIG. 6 is an explanatory view for describing a point L. FIG. 6 illustrates the same cross section as FIG. 4. FIG. 7 is an enlarged view of a part B in FIG. 6. A line M indicated by a virtual line is a line connecting the edge 513 of the light incident surface and an edge 572 on the distal end side of the first chamfer 571. The point L is an intersection of the line M and the optical axis of the illumination optical system.

As illustrated in FIG. 6, the point L is located inside the insertion unit 20, that is, to be closer to the insertion unit 20 than the distal end surface 561.

Since the observation window 51 and the illumination window 52 are arranged to have the above relationship, it is possible to prevent the illumination light emitted from the illumination window 52 from entering the observation window 51. Therefore, it is possible to provide the endoscope 10 in which flare hardly occurs.

Furthermore, the arrangements of the observation window 51 and the illumination window 52 capable of reliably preventing the occurrence of flare will be described. A distance between the distal end surface 561 and the point L is indicated by a symbol L. Further, an angle formed between the line M and the optical axis of the illumination optical system is indicated by a symbol 8.

It is desirable that 9 be 60 degrees or larger and smaller than 90 degrees. It is desirable that L be a positive value of 2 millimeters or less.

Since the meniscus lens in which the front surface side, that is, the incident surface side of the observation light, is the convex surface is used as the observation window 51 as described with reference to FIG. 3 in the present embodiment, it is possible to provide the endoscope 10 with the wide viewing angle of the observation field of view.

Further, since the illumination window 52 is the convex lens as described with reference to FIG. 3, it is possible to provide the endoscope 10 with the wide irradiation angle of the illumination light.

Incidentally, it is desirable that the irradiation range of the illumination light be slightly wider than the range of the observation field of view. As a result, it is possible to provide the endoscope 10 which can observe even a peripheral part of the field of view. Furthermore, it is possible to provide the endoscope with a high utilization efficiency of illumination light by avoiding the irradiation of the illumination light on a range out of the observation field of view.

According to the present embodiment, it is possible to provide the endoscope 10 which achieve both the wide range of the observation field of view and the prevention of flare. According to the present embodiment, it is also possible to provide the endoscope 10 which achieve both the high efficiency of illumination light and the prevention of flare. According to the present embodiment, it is also possible to provide the endoscope 10 capable of illuminating and observing a wide range.

Next, a relationship between the observation window 51, and the air supply nozzle 53 and the water supply nozzle 54 will be described. When each protruding amount of the air supply nozzle 53 and the water supply nozzle 54 from the distal end surface 561 of the endoscope 10 is too large, there is a case where illumination light is reflected to cause flare in the observation field of view. On the other hand, when the protruding amount is too small, there is a case where it is difficult to sufficiently remove a body fluid and the like which has adhering on the observation window 51. In either case, a failure occurs in observation using the endoscope 10.

Incidentally, the water supply nozzle 54 will be described hereinafter as an example, but the air supply nozzle 53 is also similar to the water supply nozzle 54.

FIG. 8 is a cross-sectional view of the distal end portion 23 taken along line VIII-VIII in FIG. 2. As described above, the water supply nozzle 54 is provided with the outlet 67 facing the observation window 51. A maximum protruding amount of the outlet, that is, a maximum protruding amount of an inner surface of the outlet of the water supply nozzle 54, is indicated by a symbol N using the distal end surface 561 as a reference. Similarly, a maximum protruding amount of an inner surface of the water supply nozzle 54 is indicated by a symbol P using the distal end surface 561 as a reference.

The protruding amount of the outlet 67 satisfies Formulas (5) and (6)

2N≥C (5)

P≥C (6)

C is the maximum protruding amount of the observation window 51 from the distal end surface 561 of the insertion unit 20.

N is the maximum protruding amount of the inner surface of the outlet 67 from the distal end surface 561 of the insertion unit 20.

P is the maximum protruding amount of the inner surface of the water supply nozzle 54 from the distal end surface 561 of the insertion unit 20.

Since the observation window 51 and the water supply nozzle 54 are arranged to have the above relationship, water emitted from the outlet 67 flows on the front surface of the observation window 51 and can remove haze of the observation window 51 and dirt or the like adhering to the observation window 51. Similarly, since the observation window 51 and the air supply nozzle 53 are arranged to have the above relationship, air emitted from the outlet 67 can blow away water droplets or the like adhering to the front surface of the observation window 51. Since the observation window 51 is cleaned during the examination as above, it is possible to provide the endoscope 10 capable of observing a clear image.

Furthermore, the arrangements of the observation window 51 and the outlet 67 which facilitate cleaning of the observation window 51 will be described. It is desirable that N be 0.3 mm or more and 0.6 mm or less. It is desirable that P be 1.5 to 2 times of N. Since N and P are defined in this manner, it is possible to suppress influence of a surface tension of water and an internal resistance of the water supply nozzle 54 and to eject water from the water supply nozzle 54 promptly according to a water supply operation performed by the user.

It is desirable that C and D have positive values, that is, the observation window 51 and the illumination window 52 protrude from the distal end surface 561 to the distal end side. This is because it is difficult to remove the attached body fluid or the like during the examination when C and D are negative values, that is, the observation window 51 and the illumination window 52 are recessed from the end surface. It is desirable that C be 0.2 mm or more and 0.5 mm or less. It is desirable that C be 0.01 mm or more and 0.3 mm or less.

Examples of preferred combinations of C, D, and N are shown in the following table. The unit is a millimeter.

TABLE 1

| Number | C | D | E | N |
|---|---|---|---|---|
| No. 1 | 0.2 | 0.01 | 0.18 | 0.3 |
| No. 2 | 0.5 | 0.3 | 0.5 | 0.3 |
| No. 3 | 0.5 | 0.1 | 0.3 | 0.6 |

Incidentally, the end surface on the distal end side of the distal end member 56 may have a step. Further, the end surface on the distal end side of the distal end member 56 may be inclined with respect to the insertion direction. In these cases, the respective protruding amounts described above are defined using, as a reference, a part of the end surface perpendicular to the insertion direction or a virtual plane perpendicular to the insertion direction defined in the vicinity of the end surface.

The endoscope 10 may be a so-called side-view or oblique-view endoscope in which the insertion direction is different from the field-of-view direction. In such a case, the respective protruding amounts described above are defined using a plane perpendicular to the optical axis of the observation optical system as a reference.

According to the present embodiment, it is possible to provide the endoscope 10 in which flare hardly occurs. Furthermore, it is possible to provide the endoscope 10 capable of cleaning the observation window 51 during the examination according to the present embodiment.

### Second Embodiment

The present embodiment relates to the endoscope 10 in which the observation window 51 is a flat plate. Descriptions regarding common parts with the first embodiment will be omitted.

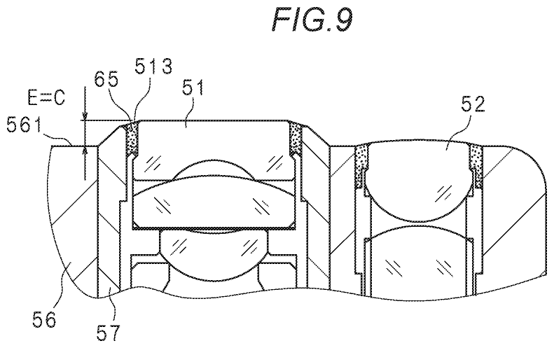

FIG. 9 is a cross-sectional view of the distal end portion 23 of the second embodiment. As illustrated in FIG. 9, the maximum protruding amount C of the observation window 51 and the protruding amount E of the edge 513 of the light incident surface of the observation window 51 coincide with each other.

According to the present embodiment, for example, a flat plate made of hard glass can be used for the observation window 51. It is possible to provide the endoscope 10 in which the observation window 51 is hardly scratched.

Technical features (constitutional requirements) described in the respective embodiments can be combined with each other, and new technical features can be formed with the combination.

The embodiments disclosed herein are exemplary in all respects, and it should be considered that the embodiments are not restrictive. The scope of the present invention is defined not by the above-described meaning but by claims, and intends to include all modifications within meaning and a scope equal to claims.

Regarding the embodiments including the first to second embodiments, the following appendixes are additionally disclosed.

### APPENDIX 1

An endoscope 10 including:

an illumination optical system that has an illumination window 52 protruding from an end surface 561 of an insertion unit 20;

a cylindrical body 57 that protrudes from the end surface 561; and

an observation window 51 that is arranged inside the cylindrical body 57 and protrudes from the end surface 561,

in which the illumination window 52 and the observation window 51 satisfy Formulas (7) and (8), and

an intersection between a straight line, which connects an edge 513 of a light incident surface of the observation window 51 and an edge 572 of an end surface of the cylindrical body 57, and an optical axis of the illumination optical system is located to be closer to the insertion unit 20 than the end surface 561 of the insertion unit 20:

C≥D (7)

E≥D (8)

where C is a maximum protruding amount of the observation window 51 from the end surface 561 of the insertion unit 20,

D is a maximum protruding amount of the illumination window 52 from the end surface 561 of the insertion unit 20, and

E is a protruding amount of the edge 513 of the light incident surface of the observation window 51 from the end surface 561 of the insertion unit 20.

### APPENDIX 2

The endoscope 10 according to Appendix 1, in which

the illumination window 52 is a convex surface.

### APPENDIX 3

The endoscope 10 according to Appendix 1 or 2, in which the observation window 51 is a convex or flat surface.

### APPENDIX 4

The endoscope 10 according to any one of Appendixes 1 to 3, in which

a distance between the intersection and the end surface 561 of the insertion unit 20 is 2 mm or less.

### APPENDIX 5

The endoscope 10 according to any one of Appendixes 1 to 4, in which

an angle formed between the straight line, which connects the edge 513 of the light incident surface of the observation window 51 and the edge of the end surface of the cylindrical body 57, and the optical axis of the illumination optical system is 60 degrees or larger and smaller than 90 degrees.

### APPENDIX 6

The endoscope 10 according to any one of Appendixes 1 to 5, in which

the cylindrical body 57 has a light shielding property.

### APPENDIX 7

The endoscope 10 according to any one of Appendixes 1 to 6, in which

a light shield 65 is provided between an inner surface of the cylindrical body 57 and the observation window 51.

### APPENDIX 8

The endoscope 10 according to any one of Appendixes 1 to 7, in which

a field-of-view direction of the observation window 51 is a longitudinal direction of the insertion unit 20.

### APPENDIX 9

The endoscope 10 according to any one of Appendixes 1 to 8, in which

the end surface 561 of the insertion unit 20 is perpendicular to the longitudinal direction of the insertion unit 20.

### APPENDIX 10

An endoscope 10 including:

an illumination window 52 that protrudes from an end surface 561 of an insertion unit 20;

an observation window 51 that protrudes from the end surface 561; and

an outlet 67 that protrudes from the end surface 561 and emits water or air to the observation window 51,

the endoscope 10 satisfying Formulas (9) and (10).

C≥D (9)

2N≥C (10)

C is a maximum protruding amount of the observation window 51 from the end surface 561 of the insertion unit 20.

D is a maximum protruding amount of the illumination window 52 from the end surface 561 of the insertion unit 20.

N is a maximum protruding amount of an inner surface of the outlet 67 from the end surface 561 of the insertion unit 20.

### APPENDIX 11

The endoscope 10 according to Appendix 10, in which the illumination window 52 is a convex surface.

### APPENDIX 12

The endoscope 10 according to Appendix 10 or claim 11, in which

the observation window 51 is a convex or flat surface.

### APPENDIX 13

The endoscope 10 according to Appendix 10, in which

the maximum protruding amount of the observation window 51 is 0.2 mm or more and 0.5 mm or less,

the maximum protruding amount of the illumination window 52 is 0.01 mm or more and 0.3 mm or less, and

the maximum protruding amount of the inner surface of the outlet 67 is 0.3 mm or more and 0.6 mm or less.

### APPENDIX 14

The endoscope 10 according to Appendix 13, in which

the maximum protruding amount of the observation window 51 is 0.3 mm or more and 0.5 mm or less.

### APPENDIX 15

The endoscope 10 according to any one of Appendixes 10 to 14, in which

a field-of-view direction of the observation window 51 is a longitudinal direction of the insertion unit 20.

### APPENDIX 16

The endoscope 10 according to any one of Appendixes 10 to 15, in which

the end surface 561 of the insertion unit 20 is perpendicular to the longitudinal direction of the insertion unit 20.

### REFERENCE SIGNS LIST

10 endoscope
20 insertion unit
21 soft portion
22 bending portion
23 distal end portion
26 bend preventing portion
40 operation unit
41 bending knob
42 channel inlet
43 forceps plug
50 connector unit
51 observation window
512 second chamfer
513 edge of light incident surface
52 illumination window
53 air supply nozzle
54 water supply nozzle
55 channel outlet
56 distal end member
561 distal end surface (end surface)
57 cylindrical body
571 first chamfer
572 edge on distal end side of first chamfer (edge of end surface of cylindrical body)
59 universal cord
61 imaging element
62 fiber bundle
63 air supply tube
64 water supply tube
65 observation bonding portion (light shield)
66 illumination bonding portion
67 outlet

1. An endoscope comprising:
an illumination optical system that has an illumination window protruding from an end surface of an insertion unit;
a cylindrical body that protrudes from the end surface; and
an observation window that is arranged inside the cylindrical body and protrudes from the end surface,
wherein the illumination window and the observation window satisfy Formulas (1) and (2), and
an intersection between a straight line, which connects an edge of a light incident surface of the observation window and an edge of an end surface of the cylindrical body, and an optical axis of the illumination optical system is located to be closer to the insertion unit than the end surface of the insertion unit:
C≥D (1)
E≥D (2)
where C is a maximum protruding amount of the observation window from the end surface of the insertion unit,
D is a maximum protruding amount of the illumination window from the end surface of the insertion unit, and
E is the protruding amount of the edge of the light incident surface of the observation window from the end surface of the insertion unit.

2. The endoscope according to claim 1, wherein
the illumination window is a convex surface.

3. The endoscope according to claim 1, wherein
the observation window is a convex or flat surface.

4. The endoscope according to claim 1, wherein
an angle formed between the straight line, which connects the edge of the light incident surface of the observation window and the edge of the end surface of the cylindrical body, and the optical axis of the illumination optical system is 60 degrees or larger and smaller than 90 degrees.

5. The endoscope according to claim 1, wherein
the cylindrical body has a light shielding property.

6. The endoscope according to claim 1, further comprising
a light shield provided between an inner surface of the cylindrical body and the observation window.

7. The endoscope according to claim 2, further comprising
a light shield provided between an inner surface of the cylindrical body and the observation window.

8. The endoscope according to claim 2, wherein
the cylindrical body has a light shielding property.

9. The endoscope according to claim 8, further comprising
a light shield provided between an inner surface of the cylindrical body and the observation window.

10. The endoscope according to claim 2, wherein
the observation window is a convex or flat surface.

11. The endoscope according to claim 10, further comprising
a light shield provided between an inner surface of the cylindrical body and the observation window.

12. The endoscope according to claim 10, wherein
the cylindrical body has a light shielding property.

13. The endoscope according to claim 12, further comprising
a light shield provided between an inner surface of the cylindrical body and the observation window.

14. The endoscope according to claim 3, further comprising
a light shield provided between an inner surface of the cylindrical body and the observation window.

15. The endoscope according to claim 3, wherein
the cylindrical body has a light shielding property.

16. The endoscope according to claim 15, further comprising
a light shield provided between an inner surface of the cylindrical body and the observation window.

17. The endoscope according to claim 4, further comprising
a light shield provided between an inner surface of the cylindrical body and the observation window.

18. The endoscope according to claim 4, wherein
the cylindrical body has a light shielding property.

19. The endoscope according to claim 18, further comprising
a light shield provided between an inner surface of the cylindrical body and the observation window.

20. The endoscope according to claim 5, further comprising
a light shield provided between an inner surface of the cylindrical body and the observation window.
